(19) United States
(12) Patent Application Publication
(10) Pub. No.: US 2025/0177059 A1
(43) Pub. Date: Jun. 5, 2025

(54) Surgical Navigation

(71) Applicants: The Board of Trustees of the Universities of Illinois, Urbana, IL (US); The Carle Foundation, Urbana, IL (US)

(72) Inventors: Alexander D. Smith, Urbana, IL (US); Kristoffer Karl Hauser, Bloomington, IN (US); Anant Naik, Eagan, MI (US); Wael Hassaneen-Mostafa, Champaign, IL (US); Paul Arnold, Champaign, IL (US)

(21) Appl. No.: 18/970,755

(22) Filed: Dec. 5, 2024

Related U.S. Application Data
(60) Provisional application No. 63/606,463, filed on Dec. 5, 2023.

(51) Int. Cl. A61B 34/20; A61B 90/10; A61B 90/50

(52) U.S. Cl. CPC A61B 34/20; A61B 90/50; A61B 2034/2059; A61B 2090/103

(57) ABSTRACT

In one aspect, a navigation device includes a plurality of link, wherein adjacent links are rotatably coupled. The navigation device includes a plurality of encoders. A base extends outwardly from a first link of the plurality of links, wherein a first encoder of the plurality of encoders is coupled to the base. A threaded bolt is rotatably coupled to the base, wherein the threaded bolt is configured to rigidly fix the base to an anatomy of a patient. A housing is coupled to a second link of the plurality of links, wherein the housing surrounds a processor, a memory, and a communications module.

### CROSS-REFERENCE TO RELATED APPLICATIONS

The present application claims the benefit of priority under 35 U.S.C. § 119 from U.S. Provisional Patent Application Ser. No. 63/606,463, entitled “Surgical Navigation,” filed on Dec. 5, 2023, all of which is incorporated herein by reference in its entirety for all purposes.

### TECHNICAL FIELD

The present disclosure generally relates to surgical devices, and more specifically to surgical navigation.

### BACKGROUND

Traumatic brain injury (TBI) disproportionately burdens low-to-middle income countries (LMIC) resulting in an estimated economic impact of $400 billion USD globally each year. External ventricular drains (EVD) are typically placed using a freehand approach, an emergency technique used in over 42,000 cases annually in the United States. Proper and timely placement of an EVD is critical to minimize adverse effects for the patient. If patients receive no treatment for increased intracranial pressure after a severe TBI or cerebrospinal fluid blockage, results are almost always fatal. Complications resulting from EVD placement can also be severe, with fatalities or permanent damage resulting from incorrect placement. Cost-effective and timely EVD placement approaches have been explored previously, but none have been successful in addressing the education and resource gaps in providing emergency TBI care in underserved areas. EVD placement surgery is a common emergency procedure performed in the emergency room or in neurosurgical operating rooms. The procedure begins by assessing a patient for the history leading to their presentation in the operating room, and an assessment of medical imaging. If a condition is observed that would result in high intracranial pressure and associated symptoms, including, but not limited to, subarachnoid hemorrhage, traumatic brain injury, or acute hydrocephalus, an EVD is a first-line treatment. Often, surgeons will opt for a freehand approach, in which they perform the procedure using external anatomical landmarks on the patient's head to guide the trajectory of the EVD based on the preoperative imaging. If the patient condition requires higher precision to hit the ventricles (i.e. “slit ventricles”), then the surgeon may opt to use surgical navigation at the cost of preparation and operating room time.

In the freehand approach, the neurosurgeon begins by identifying the ipsilateral medial canthus and ipsilateral tragus on a preoperative imaging of the patient. Typically, the patient is prepared for surgery by laying supine with the bed positioned at 45°. The patient's scalp near Kocher's point is washed with soap and water, dried, and the hair is shaved as needed. A three centimeter pen marking is made 11 centimeters posterior to the nasion and three centimeters lateral to the midline of the skull, and anterior to the motor cortex to avoid eloquent regions of the brain. The patient's head is then held by a supporting staff or secured to the bed.

The sterile site is then prepared by draping the surgical site and equipment table. The cranial access kit and an EVD are opened onto the sterile areas. Antiseptic chlorhexidine solution is used to clean the area around the marked incision sites. Local anesthetic is then injected, followed by incision to bone along the marking. A retractor holds the scalp open, and a burr hole is created using the cranial drill. The EVD is assembled by inserting the stylet into the catheter. The dura is then pierced with a spinal needle, and the EVD is advanced approximately perpendicular to the skull toward the ipsilateral medial canthus and ipsilateral tragus into the frontal horn of the ipsilateral lateral ventricle. The EVD is then checked for appropriate drainage by removing the stylet, and if drainage of cerebrospinal fluid is not achieved, then the catheter is withdrawn about 3 centimeters, and aimed toward the contralateral lateral ventricle.

Once adequate drainage is achieved, the distal end of the catheter is secured to the skin using a trocar and suture, and then attached to a collection system and intracranial pressure monitor. Finally, an antibiotic wound dressing is applied and the patient is monitored in the intensive care unit.

These procedures can additionally be augmented with stereotactic navigation, in which cameras or an electromagnetic field is co-registered with the patient's imaging, and tool position can be measured with those sensors. The EVD is attached to tools coordinated by the cameras or electromagnetic fields, and its position can be estimated relative to the patient's anatomy in real-time.

Existing devices focus on guiding EVD placement to increase precision. These devices incorporate technologies in the domains of image-guided assistance, including preoperative computed tomography (CT)-based stereotaxy with optical and magnetic resonance systems, freehand, mechanical guide-assisted, ultrasound-based with preoperative imaging, and systems that do not rely on preoperative imaging. No approved systems are currently available on the market designed for regions without access to some form of preoperative diagnostic medical imaging, which encompasses the regions most affected by the morbidity and mortality associated with TBI. Additionally, none of the existing systems provide realtime tool navigation for use outside of the operating room, relying on anatomic or geometric motion constraints.

EVD placement has highly variable accuracy rates according to the previous literature—about 73% across all freehand and navigated approaches combined based on a recent systematic review. Systems for improving the accuracy of EVD placement, especially in underserved environments, have been attempted, but these attempts have failed largely due to high costs, an inability of these devices to provide real-time feedback to the operators, or lacking infrastructure in these regions. Despite the innovation in this field, there is still a great need to improve access to TBI care on the global scale, particularly in regions without emergency access to neurosurgeons. There has been a longstanding need to develop a simple, intuitive, and safe device to assist any provider with performing emergency surgery in diagnosed severe intracranial pressure.

The description provided in the background section should not be assumed to be prior art merely because it is mentioned in or associated with the background section. The background section may include information that describes one or more aspects of the subject technology.

### SUMMARY

In certain aspects, the disclosed technology provides surgical navigation systems and methods. In certain embodiments, a surgical navigation system includes a kinematic navigation system and/or a registration system that performs a registration procedure. In some aspects, the kinematic navigation system includes five rigid links connected in series by four rotary joints, which allows for 4 degrees of freedom of motion of the distal end relative to a base, called the kinematic arm. In certain aspects, the base of the kinematic navigation system is a bone-fixation screw that attaches directly to a skull of a patient at a 10 mm burr hole, and an effector interfaces directly with an EVD. The motion of the kinematic navigation system can be visualized on a laptop screen in 3D and canonical 2D views relative to a medical image. In some aspects, a probe is attached to the distal link of the kinematic arm allowing surface features of the patient's skin to be collected or monitored as points relative to a local coordinate system.

According to certain aspects of the present disclosure, a navigation device includes a plurality of link, wherein adjacent links are rotatably coupled. The navigation device includes a plurality of encoders. A base extends outwardly from a first link of the plurality of links, wherein a first encoder of the plurality of encoders is coupled to the base. A threaded bolt is rotatably coupled to the base, wherein the threaded bolt is configured to rigidly fix the base to an anatomy of a patient. A housing is coupled to a second link of the plurality of links, wherein the housing surrounds a processor, a memory, and a communications module. In certain aspects, a third link of the plurality of links is rotatably coupled to the second link and to a third link of the plurality of links, wherein a fourth link of the plurality of links is rotatably coupled to the third link, wherein an effector extends from the fourth link, wherein a bore is disposed through the effector, wherein the bore is configured to receive an external ventricular drain. In certain aspects, the navigation device includes a probe supported by the effector. In certain aspects, the probe comprises a ball plunger switch. In certain aspects, each encoder of the plurality of encoders is in communication with the processor, and the ball plunger switch of the probe is in communication with the processor. In certain aspects, the communications module is configured to transmit data collected by the plurality of encoders and the ball plunger switch to an app on a computing device. In certain aspects, the plurality of links are rotatably coupled to provide four degrees of freedom of motion about the threaded bolt.

According to certain aspects of the present disclosure, a navigation device is provided. The navigation device includes a first link rotatably coupled to a second link at a first joint. The navigation device includes a third link rotatably coupled to the second link at a second joint. The navigation device includes a fourth link rotatably coupled to the third link at a third joint. The navigation device includes a first encoder coupled to the first joint. The navigation device includes a second encoder coupled to the second joint. The navigation device includes a third encoder coupled to the third joint. The navigation device includes a base extending from the first link. The navigation device includes a fourth encoder coupled to the base. The navigation device includes a threaded bolt rotatably coupled to the base, wherein the threaded bolt is configured to rigidly fix the base to an anatomy of a patient. The navigation device includes a housing coupled to a second link of the plurality of links, wherein the housing surrounds a processor, a memory, and a communications module. In certain aspects, the navigation device further includes an effector extending from the fourth link, wherein a bore is disposed through the effector, wherein the bore is configured to receive an external ventricular drain. In certain aspects, the navigation device further includes a probe supported by the effector. In certain aspects, the probe comprises a ball plunger switch. In certain aspects, the first encoder, the second encoder, the third encoder, and the fourth encoder are in communication with the processor, and the ball plunger switch of the probe is in communication with the processor. In certain aspects, the communications module is configured to transmit data collected by the first encoder, the second encoder, the third encoder, the fourth encoder, and the ball plunger switch to an app on a computing device. In certain aspects, the first link is rotatably coupled to the second link, the third link is rotatably coupled to the second link, and the fourth link is rotatably coupled to the third link to collectively provide four degrees of freedom of motion about the threaded bolt.

According to certain aspects of the present disclosure, method of surgical navigation is provided. The method includes receiving a scan of preoperative medical imaging of a patient. The method includes receiving, from a navigation device, location data collected by a ball plunger switch. The method includes receiving, from a navigation device, position data collected by a plurality of encoders of the navigation device. The method includes generating a point cloud representation based on the location data and the position data received. The method includes mapping the point cloud to the scan of the preoperative medical imaging of the patient. The method includes displaying a 3D visualization of the navigation device. The method includes verifying registration of the navigation device based on determining that a comparison of the point cloud and physical position of the navigation device matches the 3D visualization of the navigation device. In certain aspects, receiving the location data collected by the ball plunger switch further comprises receiving the location data when the plunger switch is depressed at a brow, a nasion, and a tip of the nose of the patient. In certain aspects, the method further includes registering the location data and the position data received via point cloud to point cloud iterative closet point. In certain aspects, the method further includes registering the location data and the position data received via point cloud to surface iterative closet point. In certain aspects, receiving the location data collected by the ball plunger switch further comprises receiving the location data when the plunger switch is depressed at a fiducial marker placed on the patient. In certain aspects, the plunger switch is depressed at the fiducial marker in a predetermined order.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 illustrates an example architecture for surgical navigation, in accordance with certain aspects of the disclosure.

FIG. 2 is a block diagram illustrating the example at least one computing device, navigation device, and in some aspects, navigation service and registration service of FIG. 1, in accordance with certain aspects of the disclosure.

FIG. 3 is a perspective view of a navigation device, in accordance with certain aspects of the disclosure.

FIG. 4 is side view of the navigation device of FIG. 3, in accordance with certain aspects of the disclosure.

FIG. 5 is a detailed end view of an effector of the navigation device of FIG. 3, in accordance with certain aspects of the disclosure.

FIG. 6 is a perspective view of a threaded bolt of the navigation device of FIG. 3 with the navigation device removed for clarity, and in use with a specialized screwdriver, in accordance with certain aspects of the disclosure.

FIG. 7 is a perspective view of the threaded bolt of the navigation device of FIG. 3 with the navigation device removed for clarity, and in use with a specialized wrench, in accordance with certain aspects of the disclosure.

FIG. 8 is a diagram illustrating a patient prepared for a surgical procedure depicting points of reference for utilization with a navigation system including the navigation device of FIG. 3, in accordance with certain aspects of the disclosure.

FIG. 9 is a diagram illustrating a medical imaging for utilization with a navigation system including the navigation device of FIG. 3, in accordance with certain aspects of the disclosure.

FIGS. 10-13 are example illustrations of screenshots of a 3D visualization of a surgical navigation procedure utilizing the navigation system, in accordance with certain aspects of the disclosure.

FIG. 14 is a diagram illustrating a surface pattern generated from a point cloud, in accordance with certain aspects of the disclosure.

FIG. 15 is an example table illustrating example measurement data, in accordance with certain aspects of the disclosure.

FIG. 16 is an example chart illustrating target registration error examples, in accordance with certain aspects of the disclosure.

FIG. 17 is an example chart illustrating time to completion registration example, in accordance with certain aspects of the disclosure.

FIG. 18 is a block diagram illustrating an example computer system with which the at least one computing device, the navigation device, and in some aspects, the navigation service and the registration service of FIG. 2 can be implemented.

In one or more implementations, not all of the depicted components in each figure may be required, and one or more implementations may include additional components not shown in a figure. Variations in the arrangement and type of the components may be made without departing from the scope of the subject disclosure. Additional components, different components, or fewer components may be utilized within the scope of the subject disclosure.

### DETAILED DESCRIPTION

The detailed description set forth below is intended as a description of various implementations and is not intended to represent the only implementations in which the subject technology may be practiced. As those skilled in the art would realize, the described implementations may be modified in various different ways, all without departing from the scope of the present disclosure. Accordingly, the drawings and description are to be regarded as illustrative in nature and not restrictive.

The disclosed technology provides surgical navigation systems that includes an intuitive device to assist any provider with performing emergency surgery in diagnosed severe intracranial pressure. The disclosed technology assists the initial medical responders in stabilizing patients with these severe neurological cases, and can greatly reduce the morbidity and mortality associated with increased intracranial pressure-causing conditions. As such, the disclosed technology advantageously provides an inexpensive real-time surgical navigation device that guides EVD placement with minimal training.

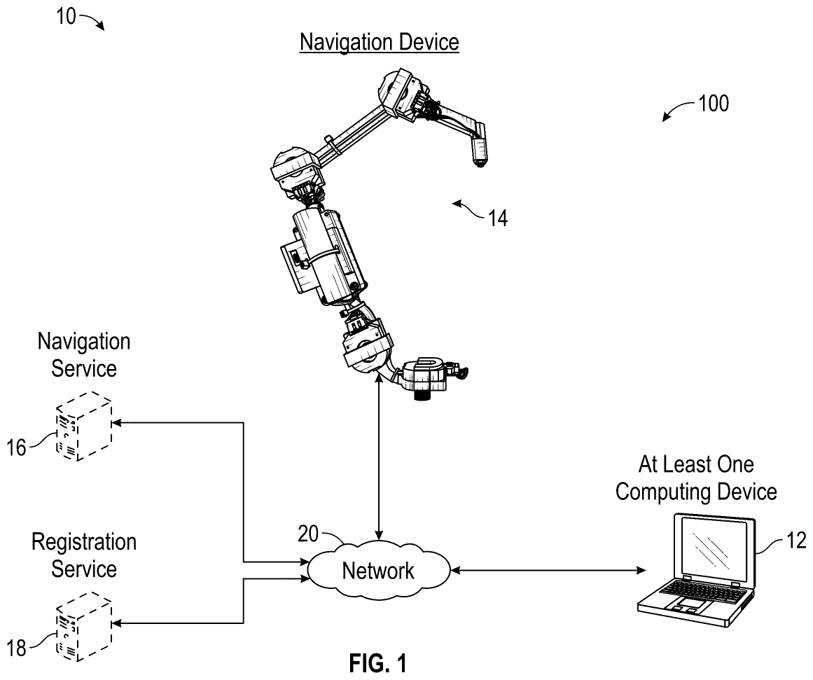

FIG. 1 illustrates an example architecture 100 of a surgical navigation system 10 for surgical navigation. For example, the architecture 100 illustrates the surgical navigation system 10, which includes at least one computing device 12, a navigation device 14, and, in certain aspects, a navigation service 16 and/or a registration service 18, all connected over a network 20.

The at least one computing device 12, to which the navigation device 14, and, in certain aspects, the navigation service 16 and/or registration service 18, communicates with over the network 20, can be, for example, a tablet computer, a mobile phone, a mobile computer, a laptop computer, a portable media player, an electronic book (eBook) reader, or any other device having appropriate processor, memory, and communications capabilities.

The navigation device 14 can be a device having an appropriate processor, memory, and communications capability for communicating with the at least one computing device 12 and, in certain aspects, the navigation service 16 and/or the registration service 18.

The navigation service 16 can be a device having an appropriate processor, memory, and communications capability for communicating with the at least one computing device 12, the navigation device 14, and, in certain aspects, the registration service 18. For purposes of load balancing, the navigation service 16 may include multiple servers. In certain aspects, the navigation service 16 can be a cloud computing server of an infrastructure-as-a-service (IaaS) and be able to support a platform-as-a-service (PaaS) and software-as-a-service (SaaS) services.

The registration service 18 can be a device having an appropriate processor, memory, and communications capability for communicating with the at least one computing device 12, the navigation device 14, and, in certain aspects, the navigation service 16. For purposes of load balancing, the navigation service 16 may include multiple servers. In certain aspects, the registration service 18 can be a cloud computing server of an infrastructure-as-a-service (IaaS) and be able to support a platform-as-a-service (PaaS) and software-as-a-service (SaaS) services.

The network 20 can include, for example, any one or more of a personal area network (PAN), a local area network (LAN), a campus area network (CAN), a metropolitan area network (MAN), a wide area network (WAN), a broadband network (BBN), the Internet, and the like. Further, the network 20 can include, but is not limited to, any one or more of the following network topologies, including a bus network, a star network, a ring network, a mesh network, a star-bus network, tree or hierarchical network, and the like.

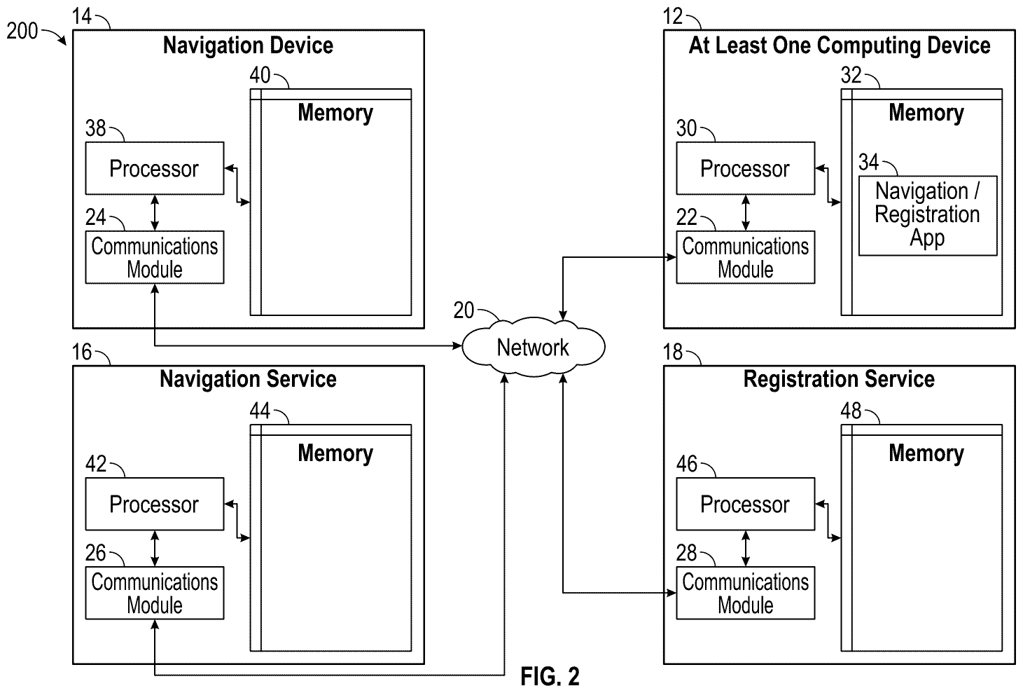

FIG. 2 is a block diagram illustrating examples of the at least one computing device 12, the navigation device 14, the navigation service 16, and the registration service 18 in the architecture 100 of FIG. 1 according to certain aspects of the disclosure. It should be understood that for purposes of explanation the at least one computing device 12 is described, but any number of the at least one computing device 12 could be used.

The at least one computing device 12, the navigation device 14, and, in certain aspects, the navigation service 16 and/or the registration service 18, are connected over the network 20 via respective communication modules 22, 24, 26, 28. The communication modules 22, 24, 26, 28 are configured to interface with the network 20 to send and receive information, such as data, requests, responses, and commands to other devices on the network 20. The communications modules 22, 24, 26, 28 can be, for example, modems or Ethernet cards.

The at least one computing device 12 includes a processor 30, the communications module 22, and a memory 32 that, in certain aspects, includes a navigation/registration app 34. Although the navigation/registration app 34 is described as one app, it should be understood that in certain aspects there could be two separate apps where one app is a navigation app and the other app is a registration app. The processor 30 of the at least one computing device 12 is configured to execute instructions, such as instructions physically coded into the processor 30, instructions received from software in memory 32, or a combination of both. In certain aspects, instead of the navigation/registration app 34, the at least one computing device 12 communicates with a navigation service 16 to perform navigation processes of the navigation/registration app 34 and/or a registration service 18 to perform registration processes of the navigation/registration app 34. Although both the navigation service 16 and the registration service 18 are described, a single service performing both navigation and registration processes could be utilized.

The navigation device 14 includes a processor 38, the communications module 24, and a memory 40. The processor 38 of the navigation device 14 is configured to execute instructions, such as instructions physically coded into the processor 38, instructions received from software in memory 40, or a combination of both.

The navigation service 16 includes a processor 42, the communications module 26, and a memory 44. The processor 42 of the navigation service 16 is configured to execute instructions, such as instructions physically coded into the processor 42, instructions received from software in memory 44, or a combination of both.

The registration service 18 includes a processor 46, the communications module 28, and a memory 48. The processor 46 of the registration service 18 is configured to execute instructions, such as instructions physically coded into the processor 46, instructions received from software in memory 48, or a combination of both.

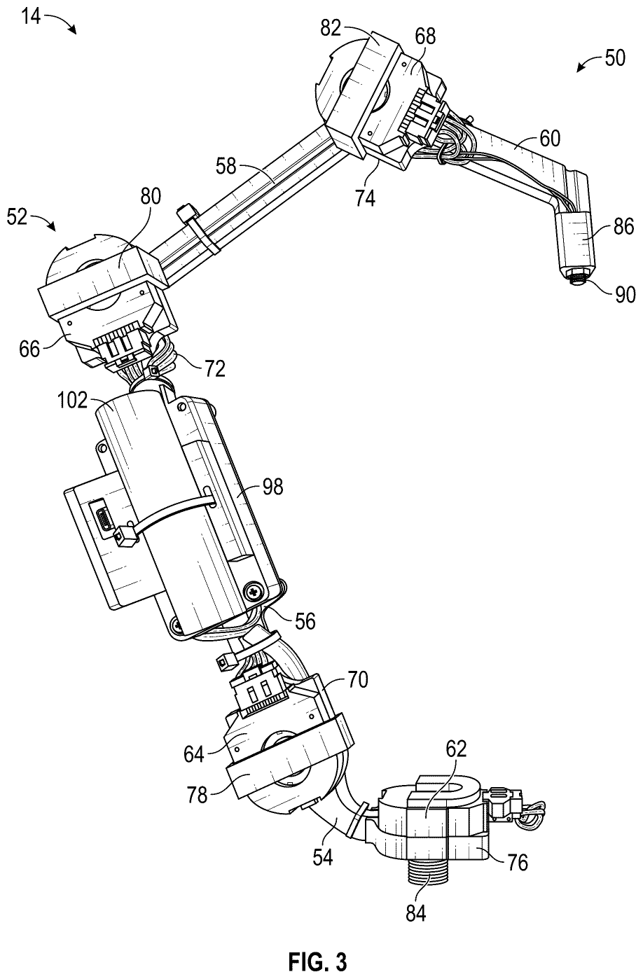

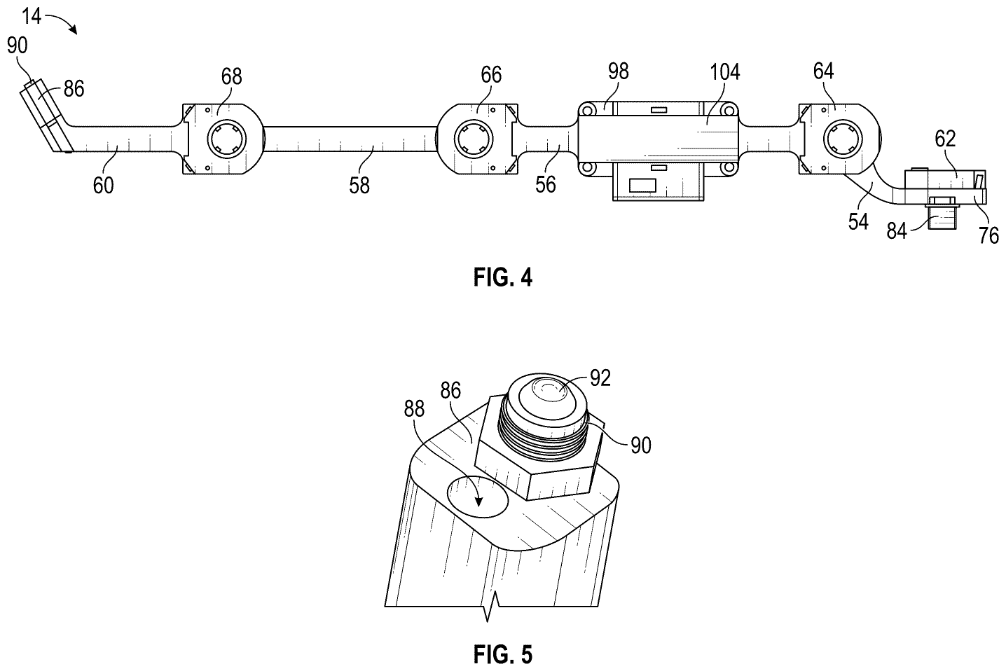

With reference to FIGS. 3-7, the navigation device 14 includes a plurality of links 50 and a plurality of encoders 52. In certain aspects, the plurality of links 50 includes a first link 54, a second link 56, a third link 58, and a fourth link 60. In certain aspects, the plurality of links 50 are machined 6063 aluminum. In certain aspects, the plurality of encoders 52 includes a first encoder 62, a second encoder 64, a third encoder 66, and a fourth encoder 68. In certain aspects, each encoder of the plurality of encoders 52 is a rotary encoder such as, but not limited to, a CUI AMT 222B-V Rotary Encoder. The first link 54 is rotatably coupled to the second link 56 at a first joint 70, the second link 54 is rotatably coupled to the third link 56 at a second joint 72, and the third link 56 is rotatably coupled to the fourth link 58 at a third joint 74. A base 76 extends outwardly from the first link 54.

The first encoder 62 is coupled to the base 76. The second encoder 64 is coupled to the first joint 70. In certain aspects, the second encoder 64 is coupled to the first joint 70 via a first adapter 78 connected to the second encoder 64 and first supporting ball bearings (not shown). The third encoder 66 is coupled to the second joint 72. In certain aspects, the third encoder 66 is coupled to the second joint 72 via a second adapter 80 connected to the third encoder 66 and second supporting ball bearings (not shown). The fourth encoder 68 is coupled to the third joint 74. In certain aspects, the fourth encoder 68 is coupled to the third joint 74 via third adapter 82 connected to the fourth encoder 68 and third supporting ball bearings (not shown). In certain aspects, a stainless steel, through-hole, threaded bolt 84 is rotatably coupled to the base 76. In certain aspects, the threaded bolt 84 is an M12 Machined stainless steel skull bolt. In certain aspects, the threaded bolt 84 is self-tapping.

A effector 86 extends outwardly from the fourth link 60. The effector 86 includes an bore 88 disposed therethrough configured to receive a stylet (not shown) and an external ventricular drain (EVD). In certain aspects, the bore 88 is approximately 3.4 mm. Both the stylet and the EVD are rigidly mounted to the effector 86 with friction when inserted into the bore 88. The EVD will slip from the effector 86 when the stylet is removed from the bore 88 due to a drop in friction between the outer surface of the EVD and the inner wall of the bore 88.

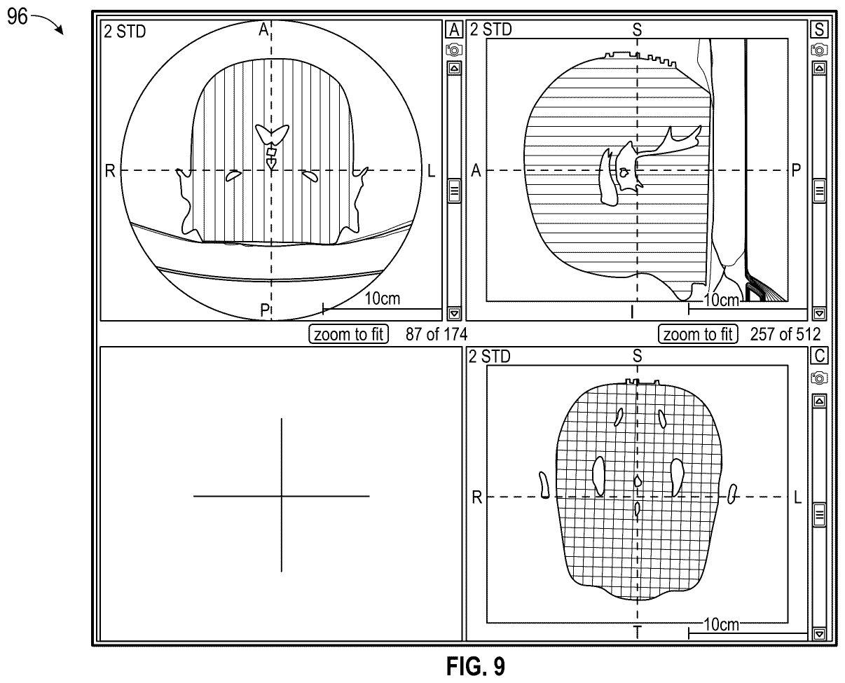

The effector 86 supports a probe 90. In certain aspects, the probe 90 includes a ball plunger switch 92 such as, but not limited to, a Misumi BP5MWA ball plunger switch. In certain aspects, the probe 90 is disposed axially on the effector 86. The ball plunger switch 92 of the probe 90 is depressible to collect features during surface or fiducial registration. The probe 90 is configured to actuate upon depression of the ball plunger switch 92. When depressed, during activation of the probe 90, the ball plunger switch 92 is configured to roll along a surface, such as a face or other body part of a patient, without snagging on skin or hair of the patient and to interface with divots of a smaller diameter than a ball of the probe 90. In the actuated state, a Cartesian position of the center of the ball plunger switch 92 relative to the base 76 of the navigation device 14 is identified and added to a point cloud 94 (see FIG. 14). The point cloud 94 is used in the registration process to map a coordinate system of the navigation device 14 to a preoperative medical imaging 96 (see FIG. 9) of the patient. Divots in a fiducial, for example, can be identified by actuating the probe 90 when the ball plunger switch 92 interfaces with the divot.

In certain aspects, the processor 38, memory 40, and communications module 24 of the navigation device 14 are supported on a printed circuit board (not shown) surrounded by a housing 98. The housing 98 is coupled to the second link 56. A battery 102 for powering the processor 38, memory 40, and communications module 24 is coupled to the housing 98. In certain aspects, the battery 102 is coupled to the exterior of the housing 98. In certain aspects, the battery 102 is a 2200 mAh battery cell. The processor 38 of the navigation device 14 is in communication with each of the first encoder 62, the second encoder 64, the third encoder 66, the fourth encoder 68, and the probe 90. In certain aspects, each of the first encoder 62, the second encoder 64, the third encoder 66, the fourth encoder 68, and the probe 90 are coupledly hard wired to the processor 38 via the printed circuit board. In certain other aspects, each of the first encoder 62, the second encoder 64, the third encoder 66, the fourth encoder 68, and the probe 90 are in wireless communication with the processor 38 of the navigation device 14.

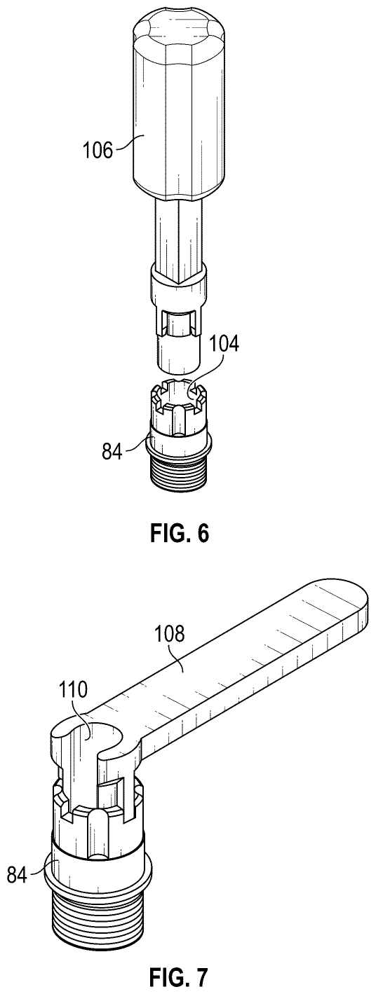

With reference to FIGS. 6 and 7, the threaded bolt 84 is a skull bolt with an inner bolt bore 104. In certain aspects, the inner bolt bore 104 is 9 mm. In certain aspects, the threaded bolt 84 includes external M12×1.7 thread. In certain aspects, the threaded bolt 84 is chamfered at its bottom edge to better tap and rigidly fix into a superficial bone cortex of the patient. As illustrated in FIG. 6, a specialized screwdriver 106 can be used to insert the threaded bolt 84 into a superficial bone cortex of the patient. A specialized wrench 108 can be used to remove the threaded bolt 84 from the superficial bone cortex of the patient. Once the threaded bolt 84 is inserted and tapped into the superficial bone cortex of the patient, the threaded bolt 84 is rigidly fixed to the superficial bone cortex of the patient such that the base 76 of the navigation device 14, which is defined as the point at the bottom of the central axis of the threaded bold 84, remains fixed relative to patient anatomy (e.g., the superficial bone cortex). With the threaded bolt 84 fixed to the superficial bone cortex, the navigation device 14 has four degrees of freedom (DOF) of motion about the threaded bolt 84. The four DOF appropriately constrains motion of an operator to allow intuitive motion, appropriate angulation, and maximal depth placement of the EVD, for example, with respect to kinematics of the navigation system 10. In certain aspects, the four DOF of the navigation device 14 of the navigation system 10 allows a workspace of approximately 28 centimeters in diameter around the threaded bolt 84 when mounted, providing sufficient reach to perform high-accuracy surface-based registration and other tasks. After the surgical procedure is completed, the threaded bolt 84 is removed from the superficial bone cortex of the patient with the specialized wrench 108 which includes a drain receiver 110, which is a cutout portion of the specialized wrench 108 that is contoured to receive the drain such that the specialized wrench 108 can fit around the drain and still operate to remove the threaded bolt 84.

While the present disclosure exemplarily describes the navigation system 10 with reference to the neurosurgical procedure of EVD placement, the navigation system 10 can be used in other skull-based trajectory oriented procedures such as, but not limited to, stereotactic biopsy, depth electrode placement, Neuralink placement, deep brain stimulation, precision laser-guided ablation, and other appropriate skull-based trajectory oriented procedures. Due to the modular nature of the navigation device 14, the navigation system 10 can be used in non-skull-based applications such as, but not limited to, spine surgery (e.g., attachment of the navigation device 14 to the pelvis for lumbar fusion, attachment to spinous processes for other fusions and tumor procedures), orthopedic surgery (e.g., fracture-reduction), plastic surgery, oncology biopsy, otolaryngology-focused targeted therapies, oral and maxillofacial (OMFS) procedures, and other appropriate applications.

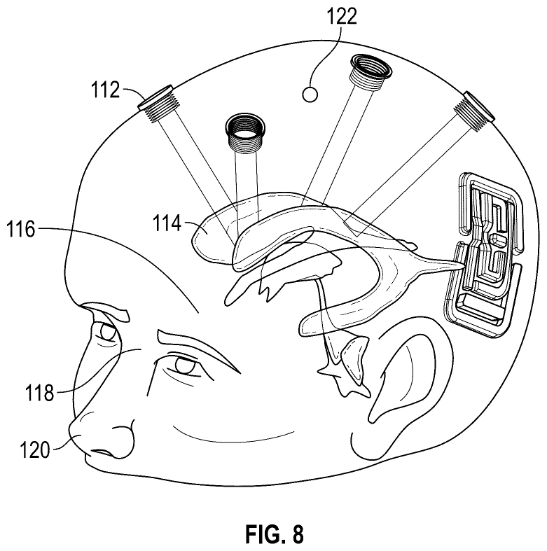

With reference to FIGS. 8-14, an exemplarily EVD placement procedure utilizing the navigation system 10 will now be described. As a brief overview, before registration, two feature sets must be acquired: one from the patient's medical image (e.g., the preoperative medical imaging 96), and one from measurements collected by the navigation device 14 and transmitted to the navigation/registration app 34 on the at least one computing device 12. Both of these feature sets must correspond to overlapping regions of the patient for registration to be successful. The operator will control the navigation device 14, guiding the probe 90 to locations based on the features intended to be collected (e.g., either surfaces in the surface-based workflow or fiducials in the fiducial-based workflow). The navigation/registration app 34 is configured to calculate the forward kinematics of the navigation device 14 based on monitored measurements of the plurality of encoders 52, updating corresponding positions of the joints of the navigation device 14 in a visualization displayed on the at least one computing device 12 via an interface of the navigation/registration app 34. The position of the point located at the center of the ball plunger switch 92 of the probe 90 is computed relative to the position of the base 76 of the navigation device 14 whenever the ball plunger switch 92 the probe 90 is depressed using a forward kinematic model. These points collected by the registration probe ball are added to the point cloud 94, which acts as the second feature set. These points are then matched to target points from the medical image feature set along the normal direction by the radius of the ball.

In a surface-based workflow, preoperative imaging (e.g., the preoperative medical imaging 96) of the patient is obtained to assess landmark features. The navigation/registration app 34 can then be opened on the at least one computing device 12 and prompt a user to perform non-sterile setup including, but not limited to, uploading the preoperative medical imaging 96, powering up the navigation device 14, connecting the navigation/registration app 34 to the navigation device 14, and setting an intended location for a selected burr hole location (e.g., Kocher's Point 112, Frazier's Point, Keen's Point). Responsive to receiving user input, the processor 30 of the at least one computing device 12 is configured to receive, via the navigation/registration app 34, a scan of the preoperative medical imaging 96. In certain aspects, the processor 30 of the at least one computing device 12 is configured to segment the preoperative medical imaging 96 (e.g., segmentation of the head and ventricles in the preoperative medical imaging 96), which acts as the first feature set.

Next, the patient and the surgical site are prepared. For example, the patient is prepared for surgery by laying supine with the bed positioned at 45 degrees. The patient's scalp near Kocher's point is washed with soap and water, dried, and the hair is shaved as needed. A three centimeter pen marking is made 11 centimeters posterior to the nasion 118 and three centimeters lateral to the midline of the skull, anterior to the motor cortex to avoid eloquent regions of the brain. The patient's head is then held by a supporting staff or secured to the bed.

The sterile site is then prepared by draping the surgical site and equipment table. The cranial access kit and an EVD 130 are opened onto the sterile areas. Antiseptic chlorhexidine solution is used to clean the area around the marked incision sites. Local anesthetic is then injected, followed by incision to bone along the marking. A retractor holds the scalp open, and a burr hole is created at Kocher's Point 112 using the cranial drill. The dura is perforated with a spinal needle. The EVD 130 is assembled and the navigation device 14 is mounted into the burr hole at Kocher's Point 112. For example, the threaded bolt 84 of the navigation device 14 is inserted into the burr hole and the specialized screwdriver 106 is used to secure the threaded bolt 84 into the burr hole until the navigation device 14 is rigidly fixed to the patient.

With the navigation device 14 rigidly secured to the patient, the user is prompted and guided via the navigation/registration app 34 on the at least one computing device 12 to perform registration (e.g., surface-based registration or fiducial-based registration) of the anatomy of the patient including pre-registration feature acquisition for generating the second feature set, coarse initialization, and fine registration. For example, with reference to coarse initialization, the user manipulates the navigation device 14 such that the ball plunger switch 92 of the probe 90 is depressed at each of the brow 116, the nasion 118, and the tip of the nose 120 of the patient. In certain aspects, the navigation/registration app 34 is configured to, based on selection of surface-based registration, a heat map of surface regions that are possible to acquire for registration (e.g., a heat map of the brow 116, the nasion 118, and the tip of the nose 120 of the patient). Responsive to each depression, the navigation/registration app 34 is configured to receive from the navigation device 14 location data of the brow 116, the nasion 118, and the tip of the nose 120 of the patient as well as position data of each encoder of the plurality of encoders 52 at the time of depression. In certain aspects, the each of encoder of the plurality of encoders 52 transmit position data at a range of approximately 250 Hz to 280 Hz with an update frequency of 60 Hz. The location data of the point located at the center of the ball plunger switch 92 is computed by the navigation/registration app 34 relative to the position data of the first encoder 62 corresponding to the base 76 of the navigation device 14 whenever the ball plunger switch 92 the probe 90 is depressed using a forward kinematic model. In certain aspects, the navigation/registration app 34 is configured to generate a mesh of the surface of the head of the patient and convert the mesh into the point cloud 94. In certain aspects, the points in the point cloud 94 are dispersed uniformly with an approximate spacing of 1-2 mm. The position data and the location data that are collected or received by the navigation/registration app 34 via the navigation device 14, which acts a second feature set, are added and aligned to the point cloud 94. The navigation/registration app 34 is configured to match the position data and the location data that are collected (e.g., the second feature set) to target points of the preoperative medical imaging 96 feature set (e.g., the first feature set) along the normal direction by the radius of the ball plunger switch 92.

The user is then prompted by the navigation/registration app 34 to trace skin surface of the patient. For example, the user manipulates the navigation device 14 to trace the surface of the patient's head with the ball plunger switch 92 depressed during the tracing such that the navigation/registration app 34 receives position data of the ball plunger switch 92 as well as location data of each encoder of the plurality of encoders 52. With the position data of the ball plunger switch 92 and the location data of each encoder of the plurality of encoders 52 that was collected during the tracing, the navigation/registration app 34 registers the data received responsive to selection of either point cloud to point cloud (PC2PC) iterative closet points (ICP) or point cloud to surface (PC2SDF) ICP.

For example, with PC2PC ICP, the navigation/registration app 34 is configured to determine corresponding closest points between the source P, representing the surface point cloud traced by the registration probe (e.g., the second feature set), and target Q, representing the sampled point cloud from the mesh, by calculating the point in Q closest to the point pᵢ in P. The navigation/registration app 34 is configured to update the transformation, applying the correspondence calculation to the transformed source point cloud P^{→}. The navigation/registration app 34 is configured to then iterate until reaching a convergence threshold or when the number of iterations has elapsed.

For example, with PC2SDF ICP, the navigation/registration app 34 is configured to convert the surface mesh of the patient anatomy to a signed distance field, and the closed points on the surface of the mesh are queried relative to each of the points in the traced point cloud (e.g., the point cloud 94). The navigation/registration app 34 is configured to then apply least-squares fitting between the queried and traced point clouds. On each iteration, the closest points between the signed distance field and the traced point cloud are resampled, and the fitting is repeated until registration error drops below a threshold or a number of iterations has elapsed.

The navigation/registration app 34 is configured to verify registration when it determines that comparison of the actual physical position of the navigation device 14 matches visualized positions of the 3D visualization of the navigation device 14 displayed on the at least one computing device 12.

As mentioned above, instead of the surface-based workflow, the fiducial-based workflow can be implemented, which follows the same path as the surface-based workflow until the registration process. With the fiducial-based workflow, the alignment of the device to the patient imaging is determined by detecting a plurality of fiducial markers (e.g., fiducial marker 122, only one shown for clarity) placed on the skin or screwed into the skull before the preoperative image is captured. In certain aspects, the navigation/registration app 34 is configured to prompt the user to enter the number of fiducial markers that are placed on the patient. Based on selection of fiducial-based registration, the user manipulates the navigation device 14 such that the ball plunger switch 92 of the probe 90 is depressed at each fiducial marker in a predetermined order. In certain aspects, the navigation/registration app 34 is configured to illuminate the current fiducial marker to be collected. Responsive to each depression, the navigation/registration app 34 is configured to receive from the navigation device 14 location data of each fiducial marker (e.g., the fiducial marker 122) as well as the position data of each encoder of the plurality of encoders 52. The navigation/registration app 34 is configured to calculate a least-squares fitting between two point sets of know correspondences. The location data of each fiducial marker that is collected or received by the navigation/registration app 34 via the navigation device 14, which acts the second feature set, are added and aligned to the point cloud 94 of the preoperative medical imaging 96. The navigation/registration app 34 is configured to verify registration when it determines that comparison of the actual physical position of the navigation device 14 matches visualized positions of the 3D visualization of the navigation device 14 displayed on the at least one computing device 12.

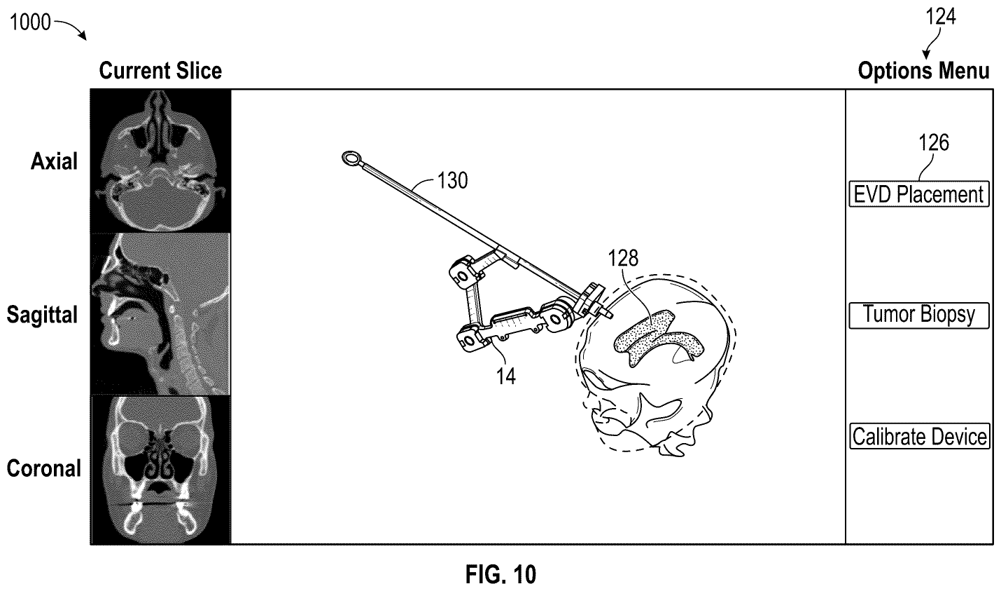

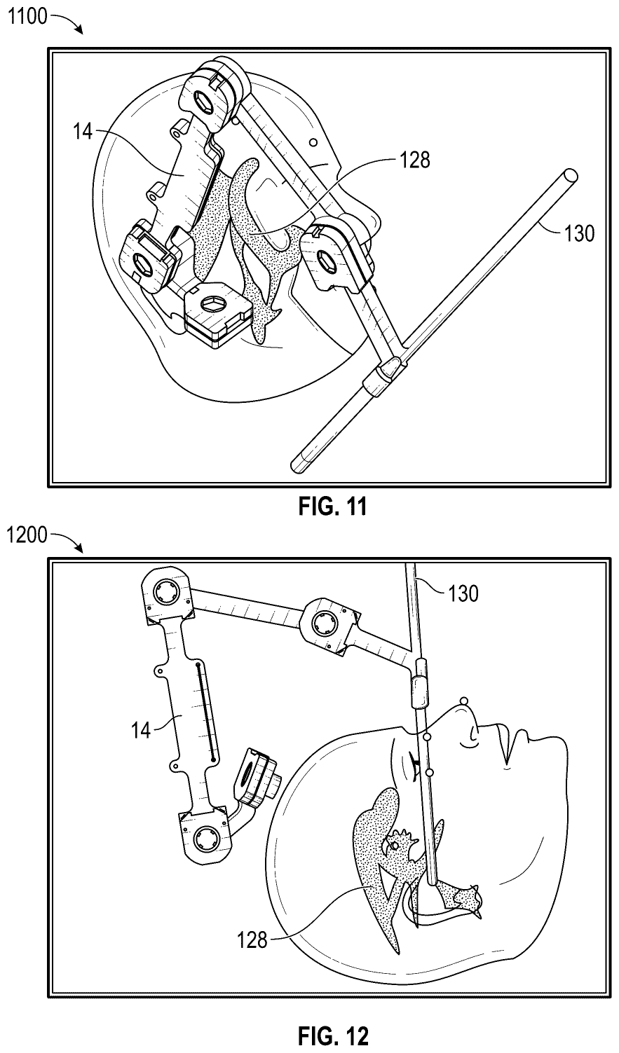

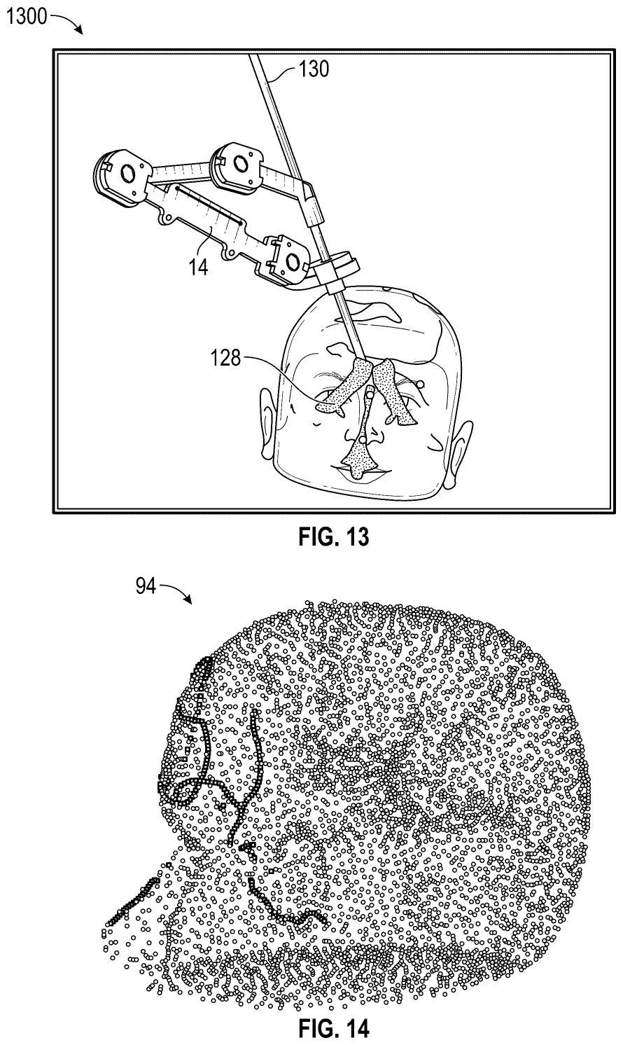

With registration of the coordinate system of the navigation device 14 to the coordinate system of the preoperative medical imaging 96, the user can place the EVD 130 into the bore 88 of the effector 86 of the navigation device 14 and view the visualization of the navigation device 14 on the at least one computing device 12 to guide the EVD 130 into the ventricle 114 of the patient, as illustrated in FIGS. 10-13. As depicted in the screenshot 11 of FIG. 11 and the screenshot 1200 of FIG. 12, because the EVD 130 is not on trajectory with the ventricle 114, the navigation/registration app 34 is configured to illuminate a heat map 128 of the ventricle 114 in first color to alert the user that the EVD 130 is not on trajectory. On the other hand, when the EVD 130 is on trajectory with the ventricle 114 the navigation/registration app 34 is configured to illuminate the heat map 128 of the ventricle a second color to alert the user that the trajectory is aligned, as depicted in the screenshot 1300 of FIG. 13. After the EVD 130 is successfully inserted into the ventricle 114, the use can remove a stylet from the EVD 130 to allow the EVD 130 to slip from the effector 86, observe drainage, and remove the navigation device 14 by using the specialized wrench 108, and slipping the navigation device 14 over the EVD 130.

In certain aspects, as illustrated in the screenshot 1000, the navigation/registration app 34 is configured to display an options menu 124, which a selection of procedures 126 can be selected, including, but not limited to EVD placement, tumor biopsy, calibrate device, and other appropriate procedures.

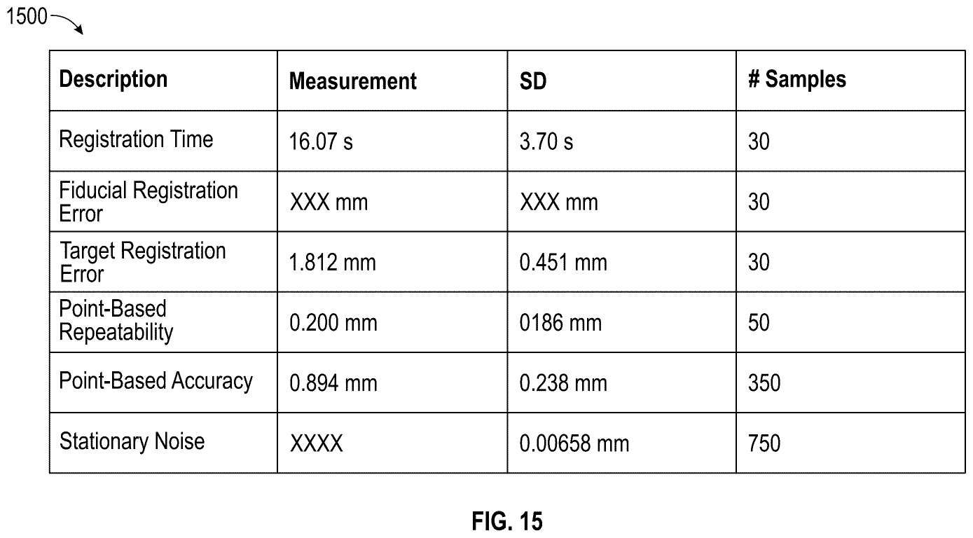

FIG. 15 is an example table illustrating example measurement data, in accordance with certain aspects of the disclosure.

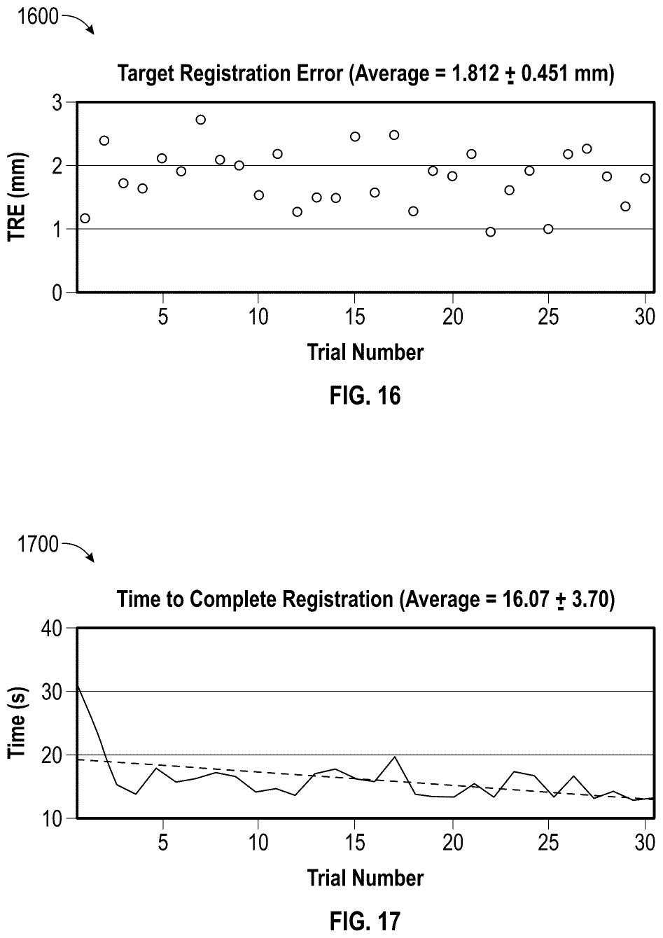

FIG. 16 is an example chart illustrating target registration error examples, in accordance with certain aspects of the disclosure.

FIG. 17 is an example chart illustrating time to completion registration example, in accordance with certain aspects of the disclosure.

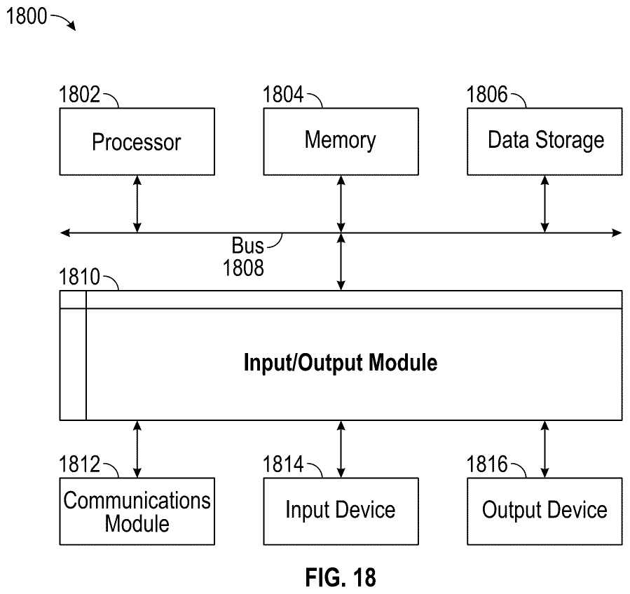

FIG. 18 is a block diagram illustrating an example computer system 1800 with which the at least one computing device 12, the navigation device 14, the navigation service 16, and the registration service 18 of FIG. 2 can be implemented. In certain aspects, the computer system 1800 may be implemented using hardware or a combination of software and hardware, either in a dedicated server, or integrated into another entity, or distributed across multiple entities.

Computer system 1800 (e.g., the at least one computing device 12, the navigation device 14, the navigation service 16, and the registration service 18) includes a bus 1808 or other communication mechanism for communicating information, and a processor 1802 (e.g., the processor 30, 38, 42, 46) coupled with bus 1808 for processing information. According to one aspect, the computer system 1800 can be a cloud computing server of an IaaS that is able to support PaaS and SaaS services.

Computer system 1800 can include, in addition to hardware, code that creates an execution environment for the computer program in question, e.g., code that constitutes processor firmware, a protocol stack, a database management system, an operating system, or a combination of one or more of them stored in an included memory 1804 (e.g., the memory 32, 40, 44, 48), such as a Random Access Memory (RAM), a flash memory, a Read Only Memory (ROM), a Programmable Read-Only Memory (PROM), an Erasable PROM (EPROM), registers, a hard disk, a removable disk, a CD-ROM, a DVD, or any other suitable storage device, coupled to bus 1808 for storing information and instructions to be executed by processor 1802. The processor 1802 and the memory 1804 can be supplemented by, or incorporated in, special purpose logic circuitry.

The instructions may be stored in the memory 1804 and implemented in one or more computer program products, e.g., one or more modules of computer program instructions encoded on a computer readable medium for execution by, or to control the operation of, the computer system 1800.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network, such as in a cloud-computing environment. The processes and logic flows described in this specification can be performed by one or more programmable processors executing one or more computer programs to perform functions by operating on input data and generating output.

Computer system 1800 further includes a data storage device 1806 such as a magnetic disk or optical disk, coupled to bus 1808 for storing information and instructions. Computer system 1800 may be coupled via input/output module 1810 to various devices. The input/output module 1810 can be any input/output module. Example input/output modules 1810 include data ports such as USB ports. In addition, input/output module 1810 may be provided in communication with processor 1802, so as to enable near area communication of computer system 1800 with other devices. The input/output module 1810 may provide, for example, for wired communication in some implementations, or for wireless communication in other implementations, and multiple interfaces may also be used. The input/output module 1810 is configured to connect to a communications module 1812. Example communications modules 1812 (e.g., the communications module 22, 24, 26, 28) include networking interface cards, such as Ethernet cards and modems.

In certain aspects, the input/output module 1810 is configured to connect to a plurality of devices, such as an input device 1814 and/or an output device 1816. Example input devices 1814 include a keyboard and a pointing device, e.g., a mouse or a trackball, by which a user can provide input to the computer system 1800. Other kinds of input devices 1814 can be used to provide for interaction with a user as well, such as a tactile input device, visual input device, audio input device, or brain-computer interface device.

According to one aspect of the present disclosure the at least one computing device 12, the navigation device 14, the navigation service 16, and the registration service 18 can be implemented using a computer system 1800 in response to processor 1802 executing one or more sequences of one or more instructions contained in memory 1804. Such instructions may be read into memory 1804 from another machine-readable medium, such as data storage device 1806. Execution of the sequences of instructions contained in main memory 1804 causes processor 1802 to perform the process steps described herein. One or more processors in a multi-processing arrangement may also be employed to execute the sequences of instructions contained in memory 1804. Processor 1802 may process the executable instructions and/or data structures by remotely accessing the computer program product, for example by downloading the executable instructions and/or data structures from a remote server through communications module 1812 (e.g., as in a cloud-computing environment). In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. For example, some aspects of the subject matter described in this specification may be performed on a cloud-computing environment. Accordingly, in certain aspects a user of systems and methods as disclosed herein may perform at least some of the steps by accessing a cloud server through a network connection. Further, data files, circuit diagrams, performance specifications and the like resulting from the disclosure may be stored in a database server in the cloud-computing environment, or may be downloaded to a private storage device from the cloud-computing environment.

The term “machine-readable storage medium” or “computer-readable medium” as used herein refers to any medium or media that participates in providing instructions or data to processor 1802 for execution. The term “storage medium” as used herein refers to any non-transitory media that store data and/or instructions that cause a machine to operate in a specific fashion. Such a medium may take many forms, including, but not limited to, non-volatile media, volatile media, and transmission media.

As used in this specification of this application, the terms “computer-readable storage medium” and “computer-readable media” are entirely restricted to tangible, physical objects that store information in a form that is readable by a computer. These terms exclude any wireless signals, wired download signals, and any other ephemeral signals. Storage media is distinct from but may be used in conjunction with transmission media. Transmission media participates in transferring information between storage media. For example, transmission media includes coaxial cables, copper wire and fiber optics, including the wires that comprise bus 1808. Transmission media can also take the form of acoustic or light waves, such as those generated during radio-wave and infra-red data communications. Furthermore, as used in this specification of this application, the terms “computer”, “server”, “processor”, and “memory” all refer to electronic or other technological devices. These terms exclude people or groups of people. For the purposes of the specification, the terms display or displaying means displaying on an electronic device.

In one aspect, a method may be an operation, an instruction, or a function and vice versa. In one aspect, a clause or a claim may be amended to include some or all of the words (e.g., instructions, operations, functions, or components) recited in either one or more clauses, one or more words, one or more sentences, one or more phrases, one or more paragraphs, and/or one or more claims.

To illustrate the interchangeability of hardware and software, items such as the various illustrative blocks, modules, components, methods, operations, instructions, and algorithms have been described generally in terms of their functionality. Whether such functionality is implemented as hardware, software or a combination of hardware and software depends upon the particular application and design constraints imposed on the overall system. Skilled artisans may implement the described functionality in varying ways for each particular application.

As used herein, the phrase “at least one of” preceding a series of items, with the terms “and” or “or” to separate any of the items, modifies the list as a whole, rather than each member of the list (e.g., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The word “exemplary” is used herein to mean “serving as an example, instance, or illustration.” Any embodiment described herein as “exemplary” is not necessarily to be construed as preferred or advantageous over other embodiments. Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

A reference to an element in the singular is not intended to mean “one and only one” unless specifically stated, but rather “one or more.” The term “some” refers to one or more. Underlined and/or italicized headings and subheadings are used for convenience only, do not limit the subject technology, and are not referred to in connection with the interpretation of the description of the subject technology. Relational terms such as first and second and the like may be used to distinguish one entity or action from another without necessarily requiring or implying any actual such relationship or order between such entities or actions. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description. No claim element is to be construed under the provisions of 35 U.S.C. § 112, sixth paragraph, unless the element is expressly recited using the phrase “means for” or, in the case of a method claim, the element is recited using the phrase “step for”.

While this specification contains many specifics, these should not be construed as limitations on the scope of what may be claimed, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted in the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products.

The title, background, brief description of the drawings, abstract, and drawings are hereby incorporated into the disclosure and are provided as illustrative examples of the disclosure, not as restrictive descriptions. It is submitted with the understanding that they will not be used to limit the scope or meaning of the claims. In addition, in the detailed description, it can be seen that the description provides illustrative examples and the various features are grouped together in various implementations for the purpose of streamlining the disclosure. The method of disclosure is not to be interpreted as reflecting an intention that the claimed subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirements of the applicable patent law, nor should they be interpreted in such a way.

What is claimed is:

1. A navigation device comprising:
a plurality of links, wherein adjacent links are rotatably coupled;
a plurality of encoders;
a base extending outwardly from a first link of the plurality of links, wherein a first encoder of the plurality of encoders is coupled to the base;
a threaded bolt rotatably coupled to the base, wherein the threaded bolt is configured to rigidly fix the base to an anatomy of a patient;
a housing coupled to a second link of the plurality of links, wherein the housing surrounds a processor, a memory, and a communications module.

2. The navigation device of claim 1, wherein a third link of the plurality of links is rotatably coupled to the second link and to a third link of the plurality of links, wherein a fourth link of the plurality of links is rotatably coupled to the third link, wherein an effector extends from the fourth link, wherein a bore is disposed through the effector, wherein the bore is configured to receive an external ventricular drain.

3. The navigation device of claim 2, further comprising a probe supported by the effector.

4. The navigation device of claim 3, wherein the probe comprises a ball plunger switch.

5. The navigation device of claim 4, wherein each encoder of the plurality of encoders is in communication with the processor, and the ball plunger switch of the probe is in communication with the processor.

6. The navigation device claim 5, wherein the communications module is configured to transmit data collected by the plurality of encoders and the ball plunger switch to an app on a computing device.

7. The navigation device of claim 1, wherein the plurality of links are rotatably coupled to provide four degrees of freedom of motion about the threaded bolt.

8. A navigation device comprising:
a first link rotatably coupled to a second link at a first joint;
a third link rotatably coupled to the second link at a second joint;
a fourth link rotatably coupled to the third link at a third joint;
a first encoder coupled to the first joint;
a second encoder coupled to the second joint;
a third encoder coupled to the third joint;
a base extending from the first link;
a fourth encoder coupled to the base;
a threaded bolt rotatably coupled to the base, wherein the threaded bolt is configured to rigidly fix the base to an anatomy of a patient;
a housing coupled to a second link, wherein the housing surrounds a processor, a memory, and a communications module.

9. The navigation device of claim 8, further comprising an effector extending from the fourth link, wherein a bore is disposed through the effector, wherein the bore is configured to receive an external ventricular drain.

10. The navigation device of claim 9, further comprising a probe supported by the effector.

11. The navigation device of claim 10, wherein the probe comprises a ball plunger switch.

12. The navigation device of claim 11, wherein the first encoder, the second encoder, the third encoder, and the fourth encoder are in communication with the processor, and the ball plunger switch of the probe is in communication with the processor.

13. The navigation device of claim 12, wherein the communications module is configured to transmit data collected by the first encoder, the second encoder, the third encoder, the fourth encoder, and the ball plunger switch to an app on a computing device.

14. The navigation device of claim 8, wherein the first link is rotatably coupled to the second link, the third link is rotatably coupled to the second link, and the fourth link is rotatably coupled to the third link to collectively provide four degrees of freedom of motion about the threaded bolt.

15. A method of surgical navigation, the method comprising:
receiving a scan of preoperative medical imaging of a patient;
receiving, from a navigation device, location data collected by a ball plunger switch;
receiving, from a navigation device, position data collected by a plurality of encoders of the navigation device;
generating a point cloud representation based on the location data and the position data received;
mapping the point cloud to the scan of the preoperative medical imaging of the patient;
displaying a 3D visualization of the navigation device; and
verifying registration of the navigation device based on determining that a comparison of the point cloud and physical position of the navigation device matches the 3D visualization of the navigation device.

16. The method of claim 15, wherein receiving the location data collected by the ball plunger switch further comprises receiving the location data when the plunger switch is depressed at a brow, a nasion, and a tip of a nose of the patient.

17. The method of claim 16, further comprising registering the location data and the position data received via point cloud to point cloud iterative closet point.

18. The method of claim 16, further comprising registering the location data and the position data received via point cloud to surface iterative closet point.

19. The method of claim 15, wherein receiving the location data collected by the ball plunger switch further comprises receiving the location data when the plunger switch is depressed at a fiducial marker placed on the patient.

20. The method of claim 19, wherein the plunger switch is depressed at the fiducial marker in a predetermined order.
